(19) United States
(12) Patent Application Publication
(10) Pub. No.: US 2010/0092546 A1
(43) Pub. Date: Apr. 15, 2010

(54) Topical and Transdermal Delivery of HIF-1 Modulators to Prevent and Treat Chronic Wounds

(76) Inventors: Geoffrey C. Gurtner, Palo Alto, CA (US); Jayakumar Rajadas, Cupertino, CA (US); Michael Gabriel Galvez, Palo Alto, CA (US); Evgenios Neofytou, Stanford, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/577,006

(22) Filed: Oct. 9, 2009

Related U.S. Application Data
(60) Provisional application No. 61/104,599, filed on Oct. 10, 2008.

(51) Int. Cl. A61K 9/70; A61K 31/164; A61K 31/44; A61K 31/4196; A61K 31/195; A61P 17/02

(52) U.S. Cl. 424/449; 514/616; 514/348; 514/383; 514/423

(57) ABSTRACT

Compositions and methods are provided for the treatment of chronic wounds, including, without limitation, pressure ulcers and diabetic ulcers, by transdermal delivery of an agent that increases activity of HIF-1α in the wound. Agents that increase HIF-1α activity include, without limitation, agents that stabilize HIF-1α, e.g. deferoxamine, deferiprone, deferasirox, etc.; agents that upregulate expression of HIF-1α, e.g. dimethyloxalylglycine, etc., HIF-1α polypeptide or coding sequences; and combinations thereof. Such agents may be referred to herein as HIF-1α potentiating agents.

### GOVERNMENT RIGHTS

This invention was made with Government support under contract AG025016 awarded by the National Institutes of Health. The Government has certain rights in this invention.

### BACKGROUND OF THE INVENTION

Nonhealing chronic wounds are a challenge to the patient, the health care professional, and the health care system. They significantly impair the quality of life for millions of people and impart burden on society in terms of lost productivity and health care dollars.

Wound healing is a dynamic pathway that optimally leads to restoration of tissue integrity and function. A chronic wound results when the normal reparative process is interrupted. By understanding the biology of wound healing, the physician can optimize the tissue environment in which the wound is present. Wound healing is the result of the accumulation of processes, including coagulation, inflammation, ground substance and matrix synthesis, angiogenesis, fibroplasia, epithelialization, wound contraction, and remodeling.

In chronic wounds, the process is disrupted, and thus healing is prolonged and incomplete. A chronic wound occurs when some factor causes the disruption of the normal, controlled inflammatory phase or the cellular proliferative phase. Thus, each wound should be evaluated to determine what factors are present and how to correct the problem. Many factors can contribute to poor wound healing. The most common include local causes such as wound infection; tissue hypoxia; repeated trauma; the presence of debris and necrotic tissue; and systemic causes such as diabetes mellitus, malnutrition, immunodeficiency, and the use of certain medications.

Wound infection, and poor circulation are common reasons for poor wound healing. Tissue perfusion may be impaired by arterial occlusion or vasoconstriction, hypotension, hypothermia, and peripheral venous congestion. Reduced wound oxygen tension can delay wound healing by slowing the production of collagen. Wound hypoxia also predisposes to bacterial infection.

Underlying systemic disease in a patient with a wound can increase the probability that the wound will become chronic. Diabetes mellitus is one example. Wound healing is often delayed because of interruption of the inflammatory and proliferative phases. Neutrophils and macrophages cannot adequately keep the bacterial load of the wound controlled, and infection prolongs the inflammatory phase. Erythrocytes can be affected by glycosylation, leading to microvascular sludging and ischemia. Low tissue oxygen tension impairs cellular proliferation and collagen synthesis.

Because chronic wounds have decreased levels of several growth factors, these have been a focus to enhance the repair of the wounds. Topically applied PDGF, TGF-β, and platelet-derived wound healing factor have been utilized in clinical trials to speed the healing of chronic wounds, and PDGF (Regranex) approved for use in the acceleration of wound closure.

Among chronic wounds are included ulcers. Ulcers are exposed surface lesions of the skin or a mucoid layer such as the lining of the mouth, where inflamed and necrotic tissue sloughs off. This exposed tissue is also highly susceptible to opportunistic microbial invasion. Infected ulcers are discomforting to the patient, disfiguring and also life-threatening if leading to a systemic infection.

Common chronic skin and soft tissue wounds include diabetic foot ulcers, pressure ulcers, and venous stasis ulcers. Diabetic ulcers are a common cause of foot and leg amputation. In patients with type I and type II diabetes, the incidence rate of developing foot ulcers is approximately 2% per year. The diabetic foot ulcer is mainly neuropathic in origin, with secondary pathogenesis being a blunted leukocyte response to bacteria and local ischemia due to vascular disease. These wounds usually occur on weight-bearing areas of the foot. Because diabetic ulcers are prone to infection, topical antimicrobials may be used if infection is present, although systemic antibiotics can eventually inhibit fibroblast and keratinocyte proliferation.

Pressure ulcers are the result of prolonged, unrelieved pressure over a bony prominence that leads to ischemia. The wound tends to occur in patients who are unable to reposition themselves to off-load weight, such as paralyzed, unconscious, or severely debilitated persons. Treatment consists of pressure relief, surgical and enzymatic debridement, moist wound care, and control of the bacterial load. Topical applications of antimicrobials and PDGF may be used.

More than 1.6 million pressure ulcers develop in the United States annually, and monetary costs are projected to reach $3.6 billion, not accounting for the impact on patient's family and quality of life. Currently, there are no options for preventing pressure ulcers and few options for improving chronic wound healing in a clinical setting. The present invention addresses this need.

### SUMMARY OF THE INVENTION

Compositions and methods are provided for the treatment of chronic wounds, including, without limitation, pressure ulcers and diabetic ulcers, by transdermal delivery of an agent that increases activity of HIF-1α in the wound. Agents that increase HIF-1α activity include, without limitation, agents that stabilize HIF-1α, e.g. deferoxamine, deferiprone, deferasirox, etc.; agents that upregulate expression of HIF-1α, e.g. dimethyloxalylglycine, etc., HIF-1α polypeptide or coding sequences; and combinations thereof. Such agents may be referred to herein as HIF-1α potentiating agents.

In some embodiments, a transdermal patch is provided, where the patch comprises a dose of a HIF-1α potentiating agent effective to increase activity of HIF-1α in the wound, and to improve wound healing. Transdermal patches may also include components such as an adhesive layer, impermeable backing membrane, release liner, transdermal delivery enhancing agents, and the like. In some embodiments the patch comprises a poloxamer gel, or polymer matrix of polyvinylpyrrolidone (PVP) and ethylcellulose, in which the active agent is entrapped.

In other embodiments, a lotion or gel is provided comprising a dose of a HIF-1α potentiating agent effective to increase activity of HIF-1α in the wound, and to improve wound healing. Such lotions or gels may further include components such as excipients, transdermal delivery enhancing agents, and the like.

In other embodiments, a method for improved healing of chronic wounds is provided, the method comprising transdermal contact of a chronic wound on an individual, with an effective dose of a HIF-1α potentiating agent, for example with a transdermal patch, lotion, gel, and the like. Methods of enhancing transdermal drug may be utilized in combination with the therapeutic composition, including, without limitation, iontophoretic and electroporation methods (applying micro-electric potential to the skin), the application of ultrasound to drive HIF potentiators into the skin, application of magnetic field as a permeation enhancer, microneedles and mechanical devices to give positive pressure, and also the use of a nano-fabricated patch with different gradients of drug loading.

### BRIEF DESCRIPTION OF THE DRAWINGS

FIGS. 1A-1C. HIF Modulators significantly prevent pressure ulcers and increase wound healing. (A) In a decubitus ulcer model, deferoxamine significantly decreases ulcer formation (ulcer grade) compared to controls. (B) Ulcer incidence in deferoxamine treated pressure ulcer model is significantly decreased (33%) compared to controls (100%) [n=6]. (C) Deferoxamine treated ulcers have earlier closure date (day 17) and smaller ulcer area compared to controls (day 19) [n=6].

FIGS. 2A-2C. Transdermal delivery of HIF-1 modulators increases HIF-1 alpha and neovascularization cytokines. (A) Increased concentrations of deferoxamine (0.1 mM to 10 mM) result in increased HIF-1 alpha stabilization compared to controls via western blot. (B) Patch for transdermal delivery, including an impermeable backing, release liner containing HIF-1 alpha modulator, and adhesive. (C) Deferoxamine significantly increases VEGF (200 ng/ml) compared to control (100 ng/ml) via ELISA.

FIGS. 3A-3C. Transdermal HIF stabilization significantly decreases reactive oxygen species, improves vascularization, and decreases cell death. (A) Superoxide staining (Dihydroethidium) is significantly increased in control ulcers compared to deferoxamine treated ulcers. (B) Vessel counts (CD31 positive staining) is significantly increased in deferoxamine treated ulcers compared to controls. (C) TUNEL staining (apoptotic cells) is significantly decreased in deferoxamine treated ulcers compared to controls.

FIG. 4. HIF Modulators improve wound healing in aged animals comparable to young controls. (A) In an established wound healing model, deferoxamine significantly improves wound healing in aged animals (day 15 closure) compared to delivery control (day 21 closure).

FIGS. 5A-5B. HIF Modulators significantly improve wound healing and neovascularization in diabetes. (A) In an established wound healing model, deferoxamine significantly improves wound healing in diabetic (Db/Db, day 13 closure) compared to delivery control (day 23 closure). (B) CD31 vessel density is significantly increased in diabetic wounds treated with deferoxamine (1000 mM) compared to delivery controls (PBS).

FIGS. 6A-6B. Transdermal delivery of HIF modulators increases HIF-1 alpha levels. (A) Patch for transdermal delivery, including an impermeable backing, release liner containing HIF-1 alpha modulator, and adhesive. (B) Deferoxamine significantly increases HIF-1 alpha, via western blot, compared to delivery control.

FIGS. 7A-7C. Topical and Transdermal delivery of HIF modulators significantly improves diabetic wound and ulcer healing. (A) Wound closure in aged animals is significantly increased with deferoxamine treatment (Day 14 closure) compared to controls. (B) Wound closure is significantly increased in diabetic animals in a dose dependent manner with deferoxamine treatment. (C) Transdermal patch delivery of deferoxamine significantly increases diabetic ulcer closure (Day 30) compared to controls (Day 45+).

### DETAILED DESCRIPTION OF THE EMBODIMENTS

The transcription factor HIF-1α is critical for new vessel formation, or neovascularization, during wound healing and has been found to be markedly impaired in chronic wounds. HIF-1α modulators are small molecules with the ability to increase HIF-1α activity, resulting in the increase of vasculogenic growth factors. By increasing neovascularization, a process central to wound healing, it is shown herein that targeted transdermal delivery of HIF-1α potentiators, e.g. through topical gels, lotions, etc. and transdermal patches can prevent and treat of chronic wounds, including ulcers such as diabetic ulcers, pressure ulcers, venous stasis ulcers, etc. Targeting the HIF-1α regulated neovascularization cascade reverses the impairments seen with aging and chronic wounds.

HIF-1α potentiators for use in the methods of the invention include small molecules that increase HIF-1α stability, such as deferoxamine and dimethyloxalylglycine. Other agents of interest increase HIF-1α activity by upregulating expression of HIF-1α, by directly providing HIF-1α activity, etc. These HIF-1 potentiators can treat and more importantly prevent a broad range of acute and chronic skin wounds in humans.

Compositions and methods are provided for the treatment of chronic wounds, including, without limitation, pressure ulcers and diabetic ulcers, by transdermal delivery of an agent that increases activity of HIF-1α in the wound. Transdermal delivery vehicles include gels, lotions, patches, etc., formulated for topical delivery.

### DEFINITIONS

The terms “treating”, and “treatment” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease, i.e., infection. The term “treatment” as used herein covers any treatment of a wound in a mammal, particularly a human, and includes: preventing a wound in an individual from dysfunction in initial healing; treating a wound that has reached a chronic state; or relieving chronic wound symptoms by mitigating or ameliorating the symptoms or conditions. The term “prophylaxis” are used herein to refer to a measure or measures taken for the prevention or partial prevention of a disease or condition.

The term “subject” includes mammals, e.g. cats, dogs, horses, pigs, cows, sheep, rodents, rabbits, squirrels, bears, primates such as chimpanzees, gorillas, and humans which are may suffer from chronic wounds, particularly chronic skin ulcers. The term “subject” also comprises elderly individuals, diabetic individuals, etc., who may be at a higher risk for chronic wounds.

The term “wound management” refers to therapeutic methods that induce and/or promote repair of a wound including, but not limited to, arresting tissue damage such as necrotization, promoting tissue growth and repair, reduction or elimination of an established microbial infection of the wound and prevention of new or additional microbial infection or colonization. The term may further include reducing or eliminating the sensation of pain attributable to a wound.

Pressure ulcers are areas of necrosis and ulceration where tissues are compressed between bony prominences and hard surfaces; they may also develop from friction and shearing forces. Risk factors include old age, impaired circulation, immobilization, malnourishment, and incontinence. Severity ranges from skin erythema to full-thickness skin loss with extensive soft-tissue necrosis. Diagnosis is clinical. Conventional treatment includes pressure reduction, avoidance of friction and shearing forces, local care, and sometimes skin grafts or myocutaneous flaps. Prognosis is excellent for early-stage ulcers; neglected and late-stage ulcers pose risk of serious infection and nutritional stress and are difficult to heal.

An estimated 1.3 to 3 million patients in the US have pressure ulcers (PUs); incidence is highest in older patients, especially when hospitalized or in long-term care facilities. Aging increases risk, in part because of reduced subcutaneous fat and decreased capillary blood flow. Immobility and comorbidities increase risk further.

Other causes of skin ulcers: Chronic arterial and venous insufficiency, e.g. associated with diabetes, can result in skin ulcers, particularly on the lower extremities. Although the underlying mechanism is vascular, the same forces that cause PUs can worsen these ulcers, and principles of treatment are similar.

Several staging systems exist; the most common classifies ulcers based on the depth of soft-tissue damage. Stage 1 ulcers manifest hyperemia, warmth, and induration. This stage is a misnomer in the sense that an ulcer (a defect of skin into the dermis) is not present. However, ulceration will form if the course is not arrested and reversed. Stage 2 ulcers involve erosion (defect of epidermis) or true ulceration; however, subcutaneous tissue is not exposed. Stage 3 and 4 ulcers have deeper involvement of underlying tissue with more extensive destruction. Patients do not always progress from lower to higher stages. Sometimes the first sign is a deep, necrotic Stage 3 or 4 ulcer. When ulcers develop quickly, subcutaneous tissue can become necrotic before the epidermis erodes. Any small ulcer should be thought of as an iceberg, with a potentially deep base.

The methods of the invention may improve the score of a skin ulcer by at least one stage, e.g. from a stage 3 or 4, to a stage 1 or 2, and may provide an improvement to where the wound is fully healed. The time required for such healing is less than the time required for healing in the absence of the treatment methods of the invention, e.g. a wound may be healed in less than about 4 weeks, less than about 3 weeks, less than about 2 weeks, or less.

Hypoxia-inducible factor (HIF-1) is an oxygen-dependent transcriptional activator, which plays crucial roles in the angiogenesis of tumors and mammalian development. HIF-1 consists of a constitutively expressed HIF-1β subunit and one of three subunits (HIF-1α, HIF-2α or HIF-3α). The stability and activity of HIF-1α are regulated by various post-translational modifications, hydroxylation, acetylation, and phosphorylation. Under normoxia, the HIF-1α subunit is rapidly degraded via the von Hippel-Lindau tumor suppressor gene product (vHL)-mediated ubiquitin-proteasome pathway. The association of vHL and HIF-1α under normoxic conditions is triggered by the hydroxylation of prolines and the acetylation of lysine within a polypeptide segment known as the oxygen-dependent degradation (ODD) domain. During hypoxic conditions HIF-1α subunit becomes stable and interacts with coactivators such as p300/CBP to modulate its transcriptional activity.

HIF-1 acts as a master regulator of numerous hypoxia-inducible genes under hypoxic conditions. The heterodimer HIF-1 binds to the hypoxic response elements (HREs) of target gene regulatory sequences, resulting in the transcription of genes implicated in the control of cell proliferation/survival, glucose/iron metabolism and angiogenesis, as well as apoptosis and cellular stress. Some of these direct target genes include glucose transporters, the glycolytic enzymes, erythropoietin, and angiogenic factor vascular endothelial growth factor (VEGF).

The term “HIF-1”, as used herein, includes both the heterodimer complex and the subunits thereof, HIF-1α and HIF-1. The HIF 1 heterodimer consists of two helix-loop-helix proteins; these are termed HIF-1α, which is the oxygen-responsive component (see, e.g., Genbank accession no. Q16665), and HIF-1β. The latter is also known as the aryl hydrocarbon receptor nuclear translocator (ARNT). Preferably, the term refers to the human form of HIF-1α (see, e.g., Genbank Accession No. NM001530).

HIF-1α may refer to any mammalian or non-mammalian protein or fragment thereof. HIF-1α gene sequences may also be obtained by routine cloning techniques, for example by using all or part of a HIF-1α gene sequence described above as a probe to recover and determine the sequence of a HIF-1α gene in another species. A fragment of HIF-1α of interest is any fragment retaining at least one functional or structural characteristic of HIF-1α.

The term “pharmaceutically acceptable” as used herein refers to a compound or combination of compounds that will not impair the physiology of the recipient human or animal to the extent that the viability of the recipient is compromised. Preferably, the administered compound or combination of compounds will elicit, at most, a temporary detrimental effect on the health of the recipient human or animal.

The term “carrier” as used herein refers to any pharmaceutically acceptable solvent of agents that will allow a therapeutic composition to be administered directly to a wound of the skin. The carrier will also allow a composition to be applied to a medical dressing for application to such a wound. A “carrier” as used herein, therefore, refers to such solvent as, but not limited to, water, saline, physiological saline, ointments, creams, oil-water emulsions, gels, or any other solvent or combination of solvents and compounds known to one of skill in the art that is pharmaceutically and physiologically acceptable to the recipient human or animal.

HIF-1α potentiating agents include agents that increase the accumulation of, or stability of, HIF-1α; directly provide HIF-1α activity; or increase expression of HIF-1. Such agents are known in the art, or may be identified through art-recognized screening methods.

A number of proteins are known to induce HIF-1α protein translation irrespective of hypoxia, including certain growth factors (see, e.g., Lee et al., Exp Mol Med 36(1):1-12 (2004), including the EBV latent membrane protein 1 (LMP1) (Wakisaka et al., Mol Cell Biol 24(12):5223-34 (2004)), and the like.

Ligands to HIF-1 form a further aspect of the invention. Agonist ligands include those that bind to the polypeptide HIF-1 or HIF-1 interacting proteins and strongly induce activity of the polypeptide and/or increases or maintain substantially the level of the polypeptide in the cell, e.g., by binding to and activating HIF-1, by binding to a nucleic acid target with which the transcription factor interacts, by facilitating or disrupting a signal transduction pathway responsible for activation of a particular regulon, and/or by facilitating or disrupting a critical protein-protein interaction.

Of particular interest are compounds currently identified as HIF-1 potentiating agents. Examples of suitable compounds include cofactor-based inhibitors such as 2-oxoglutarate analogues, ascorbic acid analogues and iron chelators such as desferrioxamine (DFO), the hypoxia mimetic cobalt chloride (CoCl₂), and mimosine, 3-Hydroxy-4-oxo-1(4H)-pyridinealanine, or other factors that may mimic hypoxia. Also of interest are hydroxylase inhibitors, including deferiprone, 2,2′-dipyridyl, ciclopirox, dimethyloxallyl glycine (DMOG), L-Mimosine (Mim) and 3-Hydroxy-1,2-dimethyl-4(1H)-Pyridone (OH-pyridone). Other HIF hydroxylase inhibitors are described herein, including but not limited to, oxoglutarates, heterocyclic carboxamides, phenanthrolines, hydroxamates, and heterocyclic carbonyl glycines (including, but not limited to, pyridine carboxamides, quinoline carboxamides, isoquinoline carboxamides, cinnoline carboxamides, beta-carboline carboxamides, including substituted quinoline-2-carboxamides and esters thereof; substituted isoquinoline-3-carboxamides and N-substituted arylsulfonylamino hydroxamic acids (see, e.g., PCT Application No. WO 05/007192, WO 03/049686 and WO 03/053997), and the like.

Compounds reported to stabilize HIF-1α also include [(3-hydroxy-6-isopropoxy-quinoline-2-carbonyl)-amino]-acetic acid, [3-hydroxy-pyridine-2-carbonyl)-amino]-acetic acid, [N-((1-chloro-4-hydroxy-isoquinoline-3-carbonyl)-amino)-acetic acid, [(7-bromo-4-hydroxy-isoquinoline-3-carbonyl)-amino]-acetic acid, [(7-chloro-3-hydroxy-quinoline-2-carbonyl)-amino]-acetic acid, [(1-bromo-4-hydroxy-7-kifluoromethyl-isoquinoline-3-carbonyl)-amino]-acetic acid, [(1-Bromo-4-hydroxy-7-phenoxy-isoquinoline-3-carbonyl)-amino]-acetic acid, [(1-Chloro-4-hydroxy-7-phenoxy-isoquinoline-3-carbonyl)-amino]-acetic acid, [(1-Chloro-4-hydroxy-7-methoxy-isoquinoline-3-carbonyl)-amino]-acetic acid, [(1-chloro-4-hydroxy-isoquinoline-3-carbonyl)-amino]-acetic acid, [(4-Hydroxy-7-phenoxy-isoquinoline-3-carbonyl)-amino]-acetic acid, [(4-Hydroxy-7-phenylsulfanyl isoquinoline-3-carbonyl)-amino]-acetic acid, [(4-Hydroxy-6-phenylsulfanyl-isoquinoline-3-carbonyl)-amino]-acetic acid, 4-oxo-1,4-dihydro-[1,10]phenanthroline-3-carboxylic acid, 4-hydroxy-5-methoxy-[1,10]phenanthroline-3-carboxylic acid ethyl ester, [(7-benzyloxy-1-chloro-4-hydroxy-isoquinoline-3-carbonyl)-amino]-acetic acid methyl ester, and 3-{[4-(3,3-Dibenzyl-ureido)-benzenesulfonyl]-[2-(4-methoxy-phenyl)-ethyl]-1-amino}-N-hydroxy-propionamide.

The HIF-1α potentiating agent or agents is formulated for dosing, typically embeddedor dispersed in a polymer, as described here. The effective dose will be determined by the selection of agent, length of time where the polymer is a biodegradable polymer intended for extended release of the drug. In general, the HIF-1α potentiating agent will be present at a concentration of at least about 1%, about 2%, about 3%, about 5% about 7.5% and not more than about 20%, not more than about 15%, not more than about 12.5%, and may at about 10%, as weight/weight percent of polymer.

The total dose of HIF-1α potentiating agent provided in a transdermal patch will be at least about 1 mg, usually at least about 5 mg, and not more than about 1000 mg, usually not more than about 500 mg, or not more than about 200 mg, and may be from about 10 mg to about 200 mg, e.g. about 100 mg.

### METHODS OF THE INVENTION

The present invention provides methods for wound management wherein a wound of a human or animal patient, e.g. a chronic skin ulcer, is contacted topically with an effective amount a therapeutic composition comprising a HIF-1α potentiating agent, and a carrier. The composition may be formulated as a patch, lotion, gel, etc., and may further comprise additional agents involved in wound healing, e.g. transdermal penetration enhancers, anti-microbial agents, and the like. Administration of the compositions of the present invention to a wound results in accelerated wound repair with reduced sepsis. Even with chronic ulcers that have penetrated the dermal layer, there is reduced pain sensation, more extensive and quicker tissue growth and less overall discomfort to the patient.

The timing of for administration a therapeutic composition of the invention, e.g. a transdermal patch, will vary for prophylaxis or treatment. The dosage of HIF modulator can determine the frequency of drug depletion in transdermal patch. For example, the transdermal patch can be applied and changed to a fresh patch every day, every other day, every third day, etc. In general it is desirable to apply a transdermal patch when a chronic wound is detected, e.g. reaches at stage 1 or stage 2, although more advanced stages will find benefit from the methods of the invention as well.

Before applying the therapeutic composition to the patient, the wound can be debrided to clean the wound of necrotic or infected tissue. Debridation may be mechanical by cutting or pulling away damaged tissue from the wound or, if readily inaccessible, other methods including, but not limited to, the application of sterile maggots may be used. Optionally, the wound may be prewashed before the application of the therapeutic composition using a composition comprising a buffering agent, detergent, etc.

The therapeutic compositions of the present invention may additionally include a pharmaceutically acceptable pH buffering agent that preferably will maintain the pH of the composition, when delivered to the skin injury or skin lesion, to between about pH 7.0 and about pH 9.0. A pH buffering agent may be selected from, but is not limited to, Tris (hydroxymethyl) aminomethane (tromethaprim; TRIZMA base), or salts thereof, phosphates or any other buffering agent such as, for example, phosphate-buffered saline that is biologically acceptable. The buffering agent may have an effective dose of between about 5 mM and about 250 mM.

The compositions of the present invention may also comprise at least one antimicrobial agent. The infections that may be treated by the methods and compositions of the present invention may be any opportunistic infection of a wound by a bacterium, or a multiple infection of more than one species of bacteria. Microbial species that may cause infections include Aerobacter aerongenes, Aeromonas spp., Bacillus spp., Bordetella spp, Campylobacter spp., Chlamydia spp., Corynebacterium spp., Desulfovibrio spp., Escherichia coli, enteropathogenic E. coli, Enterotoxin-producing E. coli, Helicobacter pylori, Klebsiella pneumoniae, Legionella pneumophiia, Leptospira spp., Mycobacterium tuberculosis, M. bovis, Neisseria gonorrhoeae, N. meningitidis, Nocardia spp., Proteus mirabilis, P. vulgaris, Pseudomonas aeruginosa, Rhodococcus equi, Salmonella enteridis, S. typhimurium, S. typhosa, Shigella sonnei, S. dysenterae, Staphylococcus aureus, Staph. epidermidis, Streptococcus anginosus, S. mutans, Vibrio cholerae, Yersinia pestis, Y. pseudotuberculosis, Actinomycetes spp., and Streptomyces spp.

The action of the antimicrobial agent can be either bacteriostatic wherein the antibiotic arrests the proliferation of, but does not necessarily kill, the microorganism or the activity of the antibiotic can be bacteriocidal and kill the organism or a combination of activities. Antibiotics suitable for use in the wound management methods of the present invention include, but are not limited to, β-lactams (penicillins and cephalosporins), vancomycins, bacitracins, macrolides (erythromycins), lincosamides (clindomycin), chloramphenicols, tetracyclines, aminoglycosides (gentamicins), amphotericns, cefazolins, clindamycins, mupirocins, sulfonamides and trimethoprim, rifampicins, metronidazoles, quinolones, novobiocins, polymixins, tetracyclines, and Gramicidins and the like and any salts or variants thereof.

The therapeutic compositions for use in the methods of wound management may also comprise a surfactant that can useful in cleaning a wound or contributing to bactericidal activity of the administered compositions. Suitable surfactants include, but are not limited to, phospholipids such as lecithin, including soy lecithin and detergents. Preferably, the surfactant selected for application to a wound or skin surface is mild and not lead to extensive irritation or promote further tissue damage to the patient.

Suitable nonionic surfactants which can be used are, for example: fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronic); fatty acid alkylolamides (fatty acid amide polyethylene glycols); alkyl polyglycosides, N-alkyl-, N-alkoxypolyhydroxy fatty acid amide, in particular N-methyl-fatty acid glucamide, sucrose esters; sorbitol esters, and esters of sorbitol polyglycol ethers. A preferred surfactant is polypropylene glycol ethoxylates with a preferred concentration of between about 5% wt % and about 25% wt %, for example Pluronic F-127 (Poloxamer 407). In other embodiments of the composition, the surfactant comprises lecithin with or without the addition of Pluronic F-127, the Pluronic F-127 being between about 2 and about 20 wt % for increasing the viscosity or gelling of the compositions.

The therapeutic compositions for use in the methods of the invention preferably include a pharmaceutically acceptable carrier that provides the medium in which are dissolved or suspended the constituents of the compositions. Suitable carriers include any aqueous medium, oil, emulsion, ointment and the like that will allow the therapeutic compositions to be delivered to the target wound without increasing damage to the tissues of the wound.

Medical dressings suitable for use in the methods of the present invention for contacting a wound with the therapeutic compositions can be any material that is biologically acceptable and suitable for placing over any chronic wound. In exemplary embodiments, the support may be a woven or non-woven fabric of synthetic or non-synthetic fibers, or any combination thereof. The dressing may also comprise a support, such as a polymer foam, a natural or man-made sponge, a gel or a membrane that may absorb or have disposed thereon, a therapeutic composition. A gel suitable for use as a support for the antimicrobial composition of the present invention is sodium carboxymethylcellulose 7H 4F.

Hydrocolloids (eg, RepliCare, DuoDERM, Restore, Tegasorb), which are combinations of gelatin, pectin, and carboxymethylcellulose in the form of wafers, powders, and pastes, are indicated for light to moderate exudate; some have adhesive backings and others are typically covered with transparent films to ensure adherence to the ulcer and must be changed q 3 days. Alginates (polysaccharide seaweed derivatives containing alginic acid), which come as pads, ropes, and ribbons (AlgiSite, Sorbsan, Curasorb), are indicated for extensive exudate and for control of bleeding after surgical debridement. Foam dressings (Allevyn, LYOfoam, Hydrasorb, Mepilex, Curafoam, Contreet) are useful as they can handle a variety of levels of exudate and provide a moist environment for wound healing. Those with adhesive backings stay in place longer and need less frequent changing.

In some embodiments, the formulation comprises permeation enhancer, e.g. transcutol, (diethylene glycol monoethyl ether), propylene glycol, dimethylsulfoxide (DMSO), menthol, 1-dodecylazepan-2-one (Azone), 2-nonyl-1,3-dioxolane (SEPA 009), sorbitan monolaurate (Span20), and dodecyl-2-dimethylaminopropanoate (DDAIP), which may be provided at a weight/weight concentration of from about 0.1% to about 10%, usually from about 2.5% to about 7.5%, more usually about 5%.

Transdermal patches may further comprise additives to prevent crystallization. Such additives include, without limitation, one or more additives selected from octyldodecanol at a concentration of from about 1.5 to about 4% w/w of polymer; dextrin derivatives at a concentration of from about 2 to about 5% w/w of polymer; polyethylene glycol (PEG) at a concentration of from about 2 to about 5% w/w of polymer; polypropylene glycol (PPG) at a concentration of from about 2 to about 5% w/w of polymer; mannitol at a concentration of from about 2 to about 4% w/w of polymer; Poloxamer 407, 188, 401 and 402 at a concentration of from about 5 to about 10% w/w of polymer; and Poloxamines 904 and 908 at a concentration of from about 2 to about 6% w/w of polymer.

In some embodiments of the invention, the HIF-1α potentiating agent is formulated in a therapeutic gel or lotion composition. The compositions of the invention include a therapeutically acceptable vehicle to act as a dilutant, dispersant or carrier, so as to facilitate its distribution and uptake when the composition is applied to the skin. Vehicles other than or in addition to water can include liquid or solid emollients, solvents, humectants, thickeners and powders.

The therapeutically acceptable vehicle will usually form 5% to 99.9%, preferably from 25% to 80% by weight of the composition, and can, in the absence of other adjuncts, form the balance of the composition.

The compositions may be in the form of aqueous, aqueous/alcoholic or oily solutions; dispersions of the lotion or serum type; anhydrous or lipophilic gels; emulsions of liquid or semi-liquid consistency, which are obtained by dispersion of a fatty phase in an aqueous phase (O/W) or conversely (W/O); or suspensions or emulsions of smooth, semi-solid or solid consistency of the cream or gel type. These compositions are formulated according to the usual techniques as are well known to this art.

When the compositions of the invention are formulated as an emulsion, the proportion of the fatty phase may range from 5% to 80% by weight, and preferably from 5% to 50% by weight, relative to the total weight of the composition. Oils, emulsifiers and co-emulsifiers incorporated in the composition in emulsion form are selected from among those used conventionally in the cosmetic or dermatological field. The emulsifer and coemulsifier may be present in the composition at a proportion ranging from 0.3% to 30% by weight, and preferably from 0.5% to 20% by weight, relative to the total weight of the composition.

When the compositions of the invention are formulated as an oily solution or gel, the fatty phase may constitute more than 90% of the total weight of the composition. Exemplary oils which may be used according to this invention include mineral oils (liquid petrolatum), plant oils (liquid fraction of karite butter, sunflower oil), animal oils (perhydrosqualen(e), synthetic oils (purcellin oil), silicone oils (cyclomethicone) and fluoro oils (perfluoropolyethers). Fatty alcohols, fatty acids (stearic acid) and waxes (paraffin wax, carnauba wax and beeswax) may also be used as fats.

Emulsifiers which may be used include glyceryl stearate, polysorbate 60, PEG-6/PEG-32/glycol stearate mixture, etc. Solvents which may be used include the lower alcohols, in particular ethanol and isopropanol, and propylene glycol.

Hydrophilic gelling agents include carboxyvinyl polymers (carbomer), acrylic copolymers such as acrylate/alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose and polyethylene.

Exemplary hydrocarbons which may serve as emollients are those having hydrocarbon chains anywhere from 12 to 30 carbon atoms. Specific examples include mineral oil, petroleum jelly, squalene and isoparaffins.

In use, a quantity of the composition, for example from 1 to 100 ml, is applied to a site of interest from a suitable container or applicator and, if necessary, it is then spread over and/or rubbed into the site using the hand or fingers or a suitable device. The product may be specifically formulated for use as a treatment for a specific area.

The lotion or gel composition of the invention can be formulated in any form suitable for application to the site of interest. The composition can be packaged in any suitable container to suit its viscosity and intended use. The invention accordingly also provides a closed container containing a therapeutically acceptable composition as herein defined.

The following examples are put forth so as to provide those of ordinary skill in the art with a complete disclosure and description of how to make and use the subject invention, and are not intended to limit the scope of what is regarded as the invention. Efforts have been made to insure accuracy with respect to the numbers used (e.g. amounts, temperature, concentrations, etc.) but some experimental errors and deviations should be allowed for. Unless otherwise indicated, parts are parts by weight, molecular weight is weight average molecular weight, temperature is in degrees centigrade, and pressure is at or near atmospheric.

### EXAMPLES

### Materials/Methods

HIF-1 alpha potentiators. HIF-1 alpha potentiators are small molecules, including those that increase HIF-1 alpha stability. Topical deferoxamine (also known as desferrioxamine, desferoxamine, DFO) was used in several concentrations (1000 mM, 500 mM, 10 mM, 1 mM, 0.1 mM) depending on experimental conditions. Additionally, a number of new iron chelators such as deferiprone, deferasirox find use. Dimethyloxalylglycine (160 mg/kg) is another HIF-1 alpha potentiator that inhibits HIF-1 alpha degradation that increases HIF-1 alpha to similar levels as deferoxamine.

Transdermal Delivery of HIF-1 alpha potentiators. A patch was designed for transdermal delivery system, including an adhesive, impermeable backing membrane, and a release liner containing HIF-1 alpha modulator (50-200 mg) dispersed or super-saturated within a biodegradable polymer. Preparation of transdermal patch includes a mixture of polymers (total weight, 400 mg, weighed in a 7:1 ratio of Ethyl Cellulose and Polyvinyl Pyrrolidone) and HIF-1 modulator drug, dissolved in 10 ml of chloroform. Additives are also included that prevent small molecule crystallization, resulting in enhanced drug release. Di-n-Butyl phthalate is then used as a plasticizer (30% weight-in-weight of polymers). To create the final release liner, this solution is then poured onto a sterile glass petri dish and dried at room temperature. The uniform dispersion, 2 ml each, is cast onto a 4% Polyvinyl Alcohol backing membrane and dried at 40 C for 6 hours. Finally, the backing membrane is attached to the contact adhesive (3M Tegaderm) keeping the matrix side upward. After 24 hours, the transdermal films are cut with a Delasco KP-16 mm circular punch biopsy and stored in a desiccator until further use.

Murine Wound Healing Model. Young (8 weeks, Jackson Laboratories) aged (18-24 months, National Institute of Aging aged rodent colony), and Diabetic (Db/Db) C57/BL6 mice (n=4 per group) underwent excisional wound biopsies in accordance with the Stanford University Institutional Animal Care and Use Committees. Wounds were made as previously described. Briefly, two 6-mm circular, full-thickness wounds were made on the dorsum of mice. A 12 mm diameter, 0.5 mm thick donut shaped silicone ring (Grace Bio-Labs, Bend, Oreg.) was then placed around the wounds preventing premature skin contracture. The silicone rings were glued to the skin with cyanoacrylate glue (Elmer's Products Inc, Columbus, Ohio) and sutured in place with 6 interrupted 6-0 nylon sutures (Ethicon Inc, Somerville, N.J.). Wounds were dressed with a sterile occlusive dressing that was changed daily, monitored, and photographed every other day until closure. Wound area was compared to the area of the inner silicone ring and reported as percentage of the original wound ratio.

Pressure Ulcer Model. Pressure ulcers on the dorsum of aged mice (19 month, NIA) and Diabetic (Db/Db) C57Bl/6 mice (n=6 per group) where created using two ceramic magnets (12 mm diameter and 5 mm thick, and average weight of 2.4 g) that apply 50 mm Hg pressure to the skin between them (Stadler et. al. J Invest Surg. 2004 July-August; 17(4):221-7). A single ischemia/reperfusion (I/R) cycle consists of placement of magnets (ischemia) for a designated time period followed by release (reperfusion). Three ischemia-reperfusion cycles were used in each animal to initiate decubitus ulcer formation (either in 3 h or 12 h cycles). Animals were housed individually, to prevent the accidental dislocation of the magnets and to prevent tampering with the resultant ulcer.

ELISA. Total protein was isolated from harvested wounds by homogenizing tissue in RIPA buffer. VEGF and SDF-1 levels were measured using the Quantikine murine VEGF and SDF-1 ELISA kits (R&D Systems, Minneapolis, Minn.) according to manufacturer's instructions.

Immunohistochemstry. CD31 staining was performed on paraffin embedded 5-micron wound sections (1:50, Santa Cruz Biotechnology, Santa Cruz, Calif.) diluted in blocking goat serum overnight at 4° C. Sections were then stained with goat anti-rat FITC secondary antibody (Santa Cruz Biotechnology, Santa Cruz, Calif.) for 1 hour at room temperature. Sections were then mounted with Vectashield plus DAPI (Vector Laboratories, Burlingame, Calif.), and analyzed using a Zeiss Axioplan 2 light-fluorescent microscope (Carl Zeiss Vision, Germany) equipped with Zeiss AxioCam HR digital imaging software (Carl Zeiss Vision). CD31+ vessel counts were performed by counting the number of capillaries present in 4 separate 40× high power fields (HPF). TUNEL (Roche) staining was also performed. All measurements were performed by two blinded observers.

Superoxide Assay (DHE). 30 μm fresh frozen sections were washed with PBS and stained with 10 μM Dihydroethidium (DHE, invitrogen) at 37 C for 30 minutes. Slides were then washed with PBS, and Vectashield with DAPI was added.

Western Blot. 50 μg of nuclear protein extract using a NE-PER kit (Pierce) and supplemented with protease inhibitor cocktail (company). Lysate protein concentrations were determined with the Micro BCA Protein Assay Kit (Pierce). Then 50 μg of nuclear lysate was fractionated by SDS-polyacrylamide gel electrophoresis (PAGE) and analyzed by immunoblotting. Protein detection was performed with primary antibodies against HIF-1α (1:500 dilution, Novus Biologicals, Littleton, Colo.) and β-actin (1:5000 dilution, Lab Vision, Fremont, Calif.) in 5%/TBS-T overnight at 4° C. Blots were then incubated with the corresponding HRP-linked secondary antibodies (1:10,000 dilution, BD Pharmingen, San Jose, Calif.) for one hour at room temperature. Blots were developed with ECL detection reagent (Amersham, UK) and exposed for 1-10 minutes using Biomax-MS film (Kodak, Rochester, N.Y.).

### Example 1

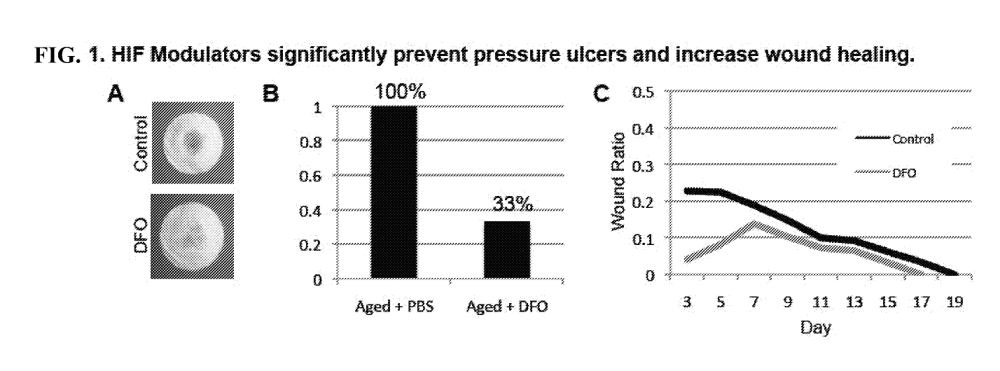

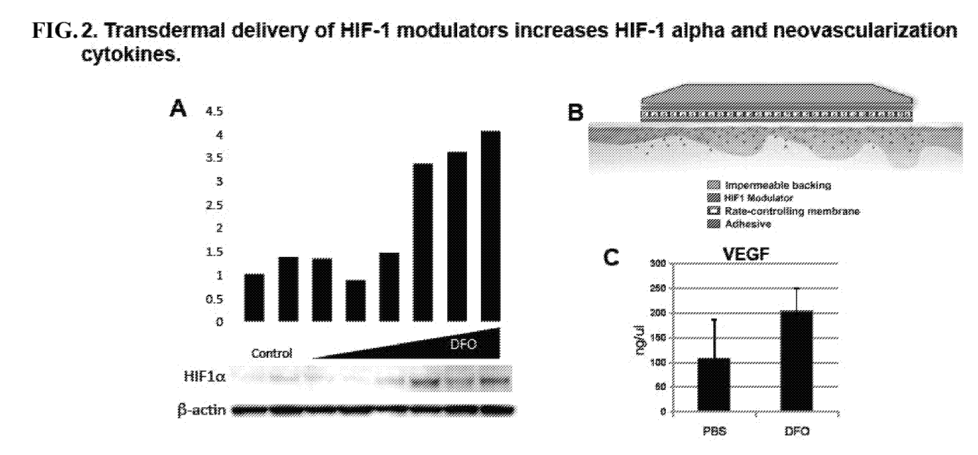

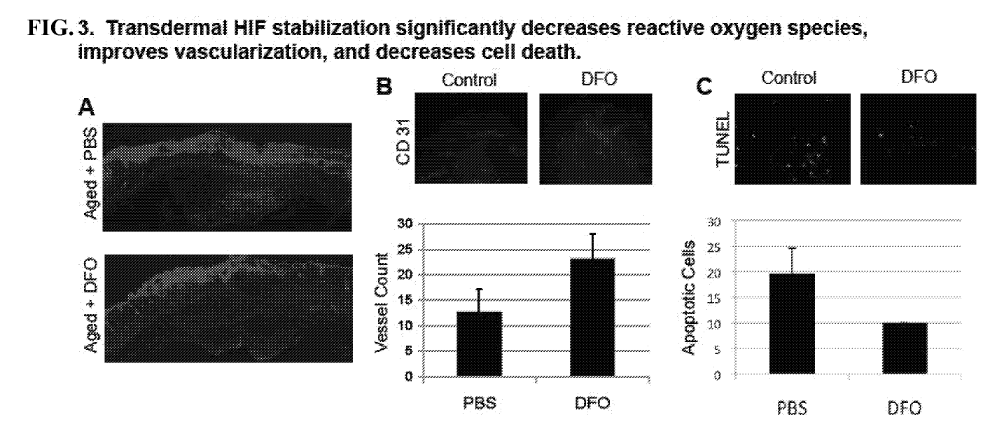

In a murine wound healing model, we have found that HIF-1 modulators act to dramatically improve healing rates and tissue survival by significantly increasing the density of blood vessels when administered topically and transdermally. In a murine pressure ulcer model, we have shown that HIF-1 alpha modulators provide an efficient and sustained means of preventing decubitus ulcer formation compared to delivery controls (FIG. 1A, 1B). Additionally, ulcer closure rates significantly increase through the correction of neovascularization (FIG. 1C). We have found that this occurs due to a dose-dependent induction of HIF-1 alpha directly and indirectly, by decreasing degradation (FIG. 2A). Induction of HIF-1 alpha increases downstream hypoxia responsive genes, which in turn decrease reactive oxygen species (FIG. 3A), stimulate vascular growth (FIG. 2C, 3B), decrease cell death (FIG. 3C), and thus improve wound healing. HIF-1 alpha modulators have promising implications for preventing ulcer formation and improving wound healing in debilitated elderly patients.

For topical delivery, deferoxamine embedded within a poloxamer gel (Pluronic F127) provides an efficient and targeted means of delivery. Hydrogels responsive to external stimuli such as pH or temperature have been studied extensively and employed for the delivery of HIF-1 alpha modulators. Because this gel can be applied topically to the wound without risks of evaporation or movement, it can deliver sustained, targeted therapy to wounds.

We have been able to characterize the biophysical properties showing effective topical delivery system for DFO including temperature and pH sensitivity, half-life, and toxicity profiles. For transdermal delivery, we have designed a transdermal patch, including an adhesive, impermeable backing membrane, and a release liner containing HIF-1 alpha modulator dispersed or super-saturated within a biodegradable polymer (FIG. 2B).

Preparation of one type of transdermal patch includes a mixture of polymers (weighed in requisite ratios of Ethyl Cellulose and Polyvinyl Pyrrolidone) and HIF-1 modulator drug, dissolved in chloroform. Additives are also included that prevent small molecule crystallization, resulting in enhanced drug release. Di-n-Butyl phthalate is then used as a plasticizer (30% weight-in-weight of polymers). To create the final release liner, this solution is then poured onto a sterile glass petri dish and dried at room temperature. The uniform dispersion is cast onto a 4% Polyvinyl Alcohol backing membrane and dried at 400 for 6 hours. Finally, the backing membrane is attached to the contact adhesive (3M Tegaderm) keeping the matrix side upward. After 24 hours, the transdermal films are cut with a Delasco KP-16 mm circular punch biopsy and stored in a desiccator until further use.

Topical application of HIF-1 modulators can be varied based on carrier agent. While Pluronic F127 is the most extensively studied poloxamer, a number of other carriers have also demonstrated clinical efficacy. *Smart hydrogels which respond to environmental stimuli such as pH and temperature have been developed to help ensure the bioactivity of drugs after delivery. Hydrogels are based on different polysaccharides, such as alginate, cellulose, chitosan, and dextran, which in turn respond to different environmental stimuli. Specifically, a chitosan based hydrogel can be manipulated to respond to temperature and pH in wound healing applications. Likewise, poloxamers such as P188 can be employed as a drug delivery gel and has demonstrated cytoprotective effects in animal models.

Transdermal patches are currently manufactured using several methods, including an adhesive, impermeable backing membrane, and a release liner. The amount of each polymer and chemicals used for patch preparation can have several modifications for maximal shelf life as well as diffusion rates.

### Example 2

Targeting the HIF-1 alpha regulated neovascularization cascade reverses the impairments seen with diabetic wounds. HIF-1 alpha modulators such as deferoxamine and dimethyloxalylglycine, are small molecules that increase HIF-1 alpha stability. Deferoxamine (also known as desferrioxamine, desferoxamine, DFO) is a FDA-approved iron chelator approved for systemic administration. Dimethyloxalylglycine inhibits HIF-1 alpha degradation, thus also increasing HIF-1 alpha levels. These HIF-1 modulators can treat and more importantly prevent a broad range of diabetic wounds and ulcers in humans.

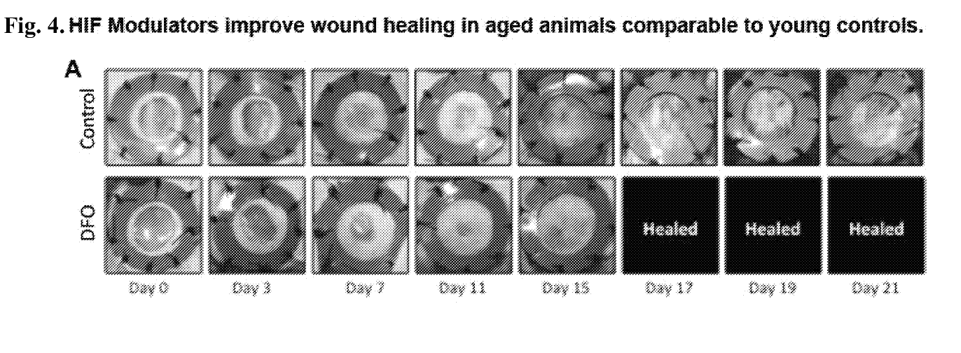

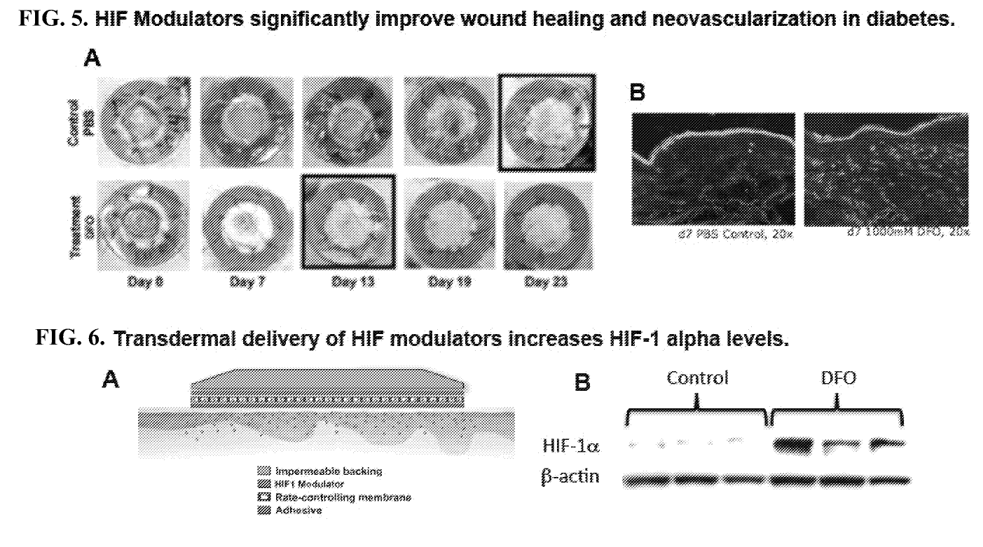

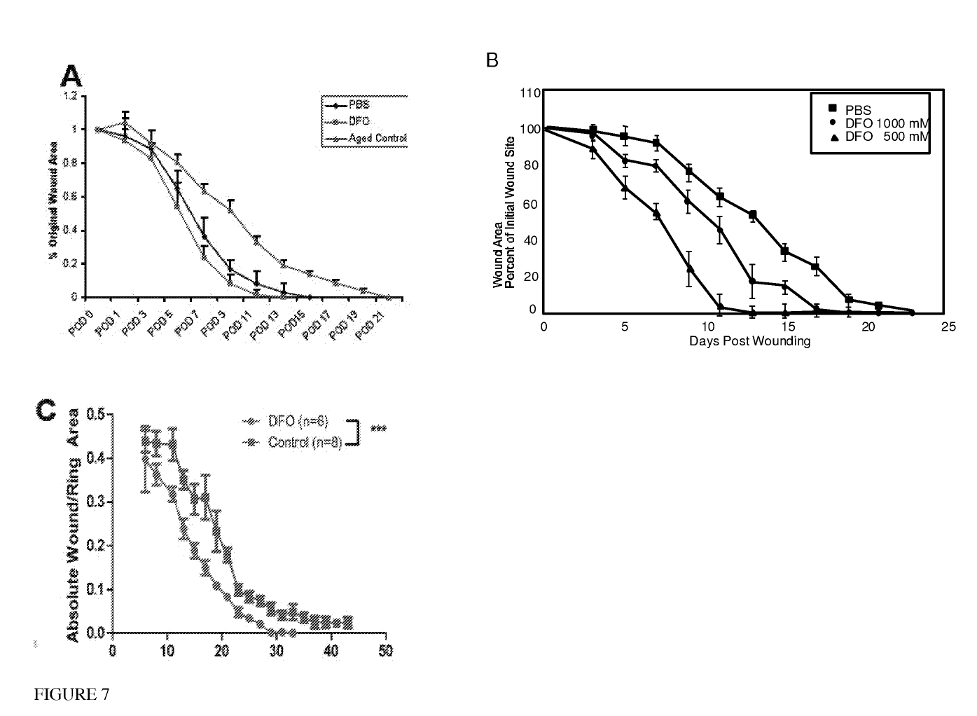

In a murine wound healing model, we have found that local delivery of HIF-1 alpha modulators act to dramatically improve healing in aged animals comparable to young controls (FIG. 4A, 7A), and in diabetic animals (FIG. 5A, 7B). Diabetic animals show markedly decreased wound healing, with wound closure at Day 23. Treatment with topical delivery of HIF-1 alpha modulators results in significantly improved wound healing, with wound closure at Day 13. Additionally, significant tissue survival is noted with the increased of blood vessel density (FIG. 2B) when administered topically and transdermally. In a murine pressure ulcer model, we have shown that transdermal delivery of HIF-1 alpha modulators provide an efficient and sustained means of treating diabetic ulcer formation compared to delivery controls (FIG. 7C).

Furthermore, we have discovered there is a dose-dependent increase in closure rates through the correction of neovascularization (FIG. 5B). We have found that this most likely occurs due to induction of HIF-1 alpha directly and indirectly, by decreasing degradation (FIG. 6B). Induction of HIF-1 alpha increases downstream hypoxia responsive genes, which stimulates an increase in vascular growth and improves wound healing. HIF-1 alpha modulators have promising implications for treating diabetic wounds and ulcers.

For topical delivery, deferoxamine embedded within a poloxamer gel (Pluronic F127) provides an efficient and targeted means of delivery. Hydrogels responsive to external stimuli such as pH or temperature have been studied extensively and employed for the delivery of HIF-1 alpha modulators. Because this gel can be applied topically to the wound without risks of evaporation or movement, it can deliver sustained, targeted therapy to wounds. We have been able to characterize the biophysical properties showing effective topical delivery system for DFO including temperature and pH sensitivity, half-life, and toxicity profiles.

For transdermal delivery, we have designed a transdermal patch, including an adhesive, impermeable backing membrane, and a release liner containing HIF-1 alpha modulator dispersed or super-saturated within a biodegradable polymer (FIG. 6A). Preparation of one type of transdermal patch includes a mixture of polymers (weighed in requisite ratios of Ethyl Cellulose and Polyvinyl Pyrrolidone) and HIF-1 modulator drug, dissolved in chloroform. Additives are also included that prevent small molecule crystallization, resulting in enhanced release of the drug. Din-Butyl phthalate is then used as a plasticizer (30% weight-in-weight of polymers). To create the final release liner, this solution is then poured onto a sterile glass petri dish and dried at room temperature. The uniform dispersion is cast onto a 4% Polyvinyl Alcohol backing membrane and dried at 40 C for 6 hours. Finally, the backing membrane is attached to the contact adhesive (3M Tegaderm) keeping the matrix side upward. After 24 hours, the transdermal films are cut with a Delasco KP-16 mm circular punch biopsy and stored in a desiccator until further use. The targeted delivery of HIF-1 alpha modulators through topical gels and transdermal patches can prevent and treat diabetic wounds and ulcers.

What is claimed is:

1. A method of treating a chronic skin wound on an individual, the method comprising:
contacting said wound topically with an effective dose of a HIF-1α potentiating agent.

2. The method of claim 1, wherein the HIF-1α potentiating agent transdermally penetrates the wound.

3. The method of claim 1, wherein the HIF-1α potentiating agent stabilizes HIF-1α.

4. The method of claim 3, wherein the agent is selected from deferoxamine, deferiprone, and deferasirox.

5. The method of claim 1, wherein the HIF-1α potentiating agent upregulates expression of HIF-1α.

6. The method of claim 5, wherein the agent is dimethyloxalylglycine.

7. The method of claim 1, wherein the chronic wound is a skin ulcer.

8. The method of claim 7, wherein the skin ulcer is a decubitus ulcer.

9. The method of claim 7, wherein the skin ulcer is a diabetic ulcer.

10. The method of claim 7, wherein the ulcer is a venous stasis ulcer.

11. The method of claim 1, wherein the HIF-1α potentiating agent is provided in a lotion or gel.

12. The method of claim 1, wherein the HIF-1α potentiating agent is provided in a transdermal patch.

13. The method of claim 12, wherein the transdermal patch comprises the HIF-1α potentiating agent embedded in a gel.

14. The method of claim 13, wherein the gel is a poloxamer gel.

15. The method of claim 12, wherein the transdermal patch comprises the HIF-1α potentiating agent embedded in a biodegradable polymer.

16. The method of claim 15, wherein the biodegradable polymer comprises one or both of ethyl cellulose and polyvinyl pyrrolidine.

17. The method of claim 12, wherein the transdermal patch comprises an adhesive; an impermeable backing membrane; and gel or polymer comprising the HIF-1α potentiating agent.

18. The method of claim 12, wherein the polymer further comprises an agent to inhibit crystallization.

19. The method of claim 12, wherein the polymer further comprises a permeation enhancer.

20. A transdermal patch for use according to the methods of claim 12.

21. A lotion or gel for use in the method of claim 11.
